SEQUENCE LISTING (5 sequences)

SEQ ID NO: 1 - protein, 12 aa, Artificial Sequence
  GAPDH binding peptide
GNLGINVTISMC

SEQ ID NO: 2 - protein, 19 aa, Artificial Sequence
  Siah1 binding peptide
VIPELNGKLTGMAFRVPTA

SEQ ID NO: 3 - protein, 11 aa, Artificial Sequence
  Cationic internalization sequence
YGRKKRRQRRR

SEQ ID NO: 4 - protein, 335 aa, Homo sapiens
MGKVKVGVNGFGRIGRLVTRAAFNSGKVDIVAINDPFIDLNYMVYMFQYDSTHGKFHGTVKAENGKLVINGNPITIFQERDPSKIKWGDAGAEYVVESTGVFTTMEKAGAHLQGGAKRVIISAPSADAPMFVMGVNHEKYDNSLKIISNASCTTNCLAPLAKVIHDNFGIVEGLMTTVHAITATQKTVDGPSGKLWRDGRGALQNIIPASTGAAKAVGKVIPELNGKLTGMAFRVPTANVSVVDLTCRLEKPAKYDDIKKVVKQASEGPLKGILGYTEHQVVSSDFNSDTHSSTFDAGAGIALNDHFVKLISWYDNEFGYSNRVVDLMAHMASKE

SEQ ID NO: 5 - protein, 282 aa, Homo sapiens
MSRQTATALPTGTSKCPPSQRVPALTGTTASNNDLASLFECPVCFDYVLPPILQCQSGHLVCSNCRPKLTCCPTCRGPLGSIRNLAMEKVANSVLFPCKYASSGCEITLPHTEKADHEELCEFRPYSCPCPGASCKWQGSLDAVMPHLMHQHKSITTLQGEDIVFLATDINLPGAVDWVMMQSCFGFHFMLVLEKQEKYDGHQQFFAIVQLIGTRKQAENFAYRLELNGHRRRLTWEATPRSIHEGIATAIMNSDCLVFDTSIAQLFAENGNLGINVTISMC

(19) United States
(12) Patent Application Publication
(10) Pub. No.: US 2018/0296639 A1
(43) Pub. Date: Oct. 18, 2018

(54) THERAPEUTIC PEPTIDES

(71) Applicant: Hoffmann-La Roche Inc., Little Falls, NJ (US)

(72) Inventor: Ashwath JAYAGOPAL, Basel (CH)

(73) Assignee: Hoffmann-La Roche Inc., Little Falls, NJ (US)

(21) Appl. No.: 15/946,701

(22) Filed: Apr. 5, 2018

Related U.S. Application Data
(63) Continuation of application No. PCT/EP2016/073531, filed on Oct. 3, 2016.

(30) Foreign Application Priority Data
Oct. 6, 2015 (EP) 15188478.0

(51) Int. Cl. A61K 38/17; A61P 27/02; A61K 38/54

(52) U.S. Cl. CPC A61K 38/1709; A61K 38/54; A61P 27/02

(57) ABSTRACT

The invention discloses peptides for the treatment and/or prophylaxis of diabetic retinopathy. The peptides of the invention comprise a glyceraldehyde-3-phosphate dehydrogenase (GAPDH) binding sequence and/or an E3 ubiquitin ligase seven in absentia homolog 1 (Siah1) binding sequence and an internalization sequence.

### CROSS REFERENCE TO RELATED APPLICATIONS

This application is a continuation of International Application No. PCT/EP2016/073531 having an international filing date of Oct. 3, 2016 and which claims benefit under 35 U.S.C. § 119 to European Patent Application No. 15188478.0 having an international filing date of Oct. 6, 2015. The entire contents of both are incorporated herein by reference.

### SEQUENCE LISTING

This application contains a Sequence Listing which has been submitted via EFS-Web and is hereby incorporated by reference in its entirety. Said ASCII copy, created on Mar. 5, 2018, is named P33109-US-sequencelisting.txt and is 7000 bytes in size.

### FIELD OF THE INVENTION

The present invention relates to peptides binding to glyceraldehyde-3-phosphate dehydrogenase (GAPDH) and/or the E3 ubiquitin ligase, seven in absentia homolog 1 (Siah1) and its use in the treatment or prophylaxis of diabetic retinopathy.

### BACKGROUND

Diabetic Retinopathy (DR) is a leading cause of blindness worldwide, and its prevalence is growing. Current therapies for DR address only the later stages of the disease, are invasive and are of limited effectiveness. Retinal pericyte death is an early pathologic feature of DR. Though it has been observed in diabetic patients and in animal models of DR, the cause of pericyte death remains unknown. A novel pro-apoptotic pathway initiated by the interaction between glycer-aldehyde-3-phosphate dehydrogenase (GAPDH) and the E3 ubiquitin ligase, seven in absentia homolog 1 (Siah1), was recently identified in ocular tissues.

The problem to be solved by the present invention was to provide new therapeutic peptides for the treatment or prophylaxis of diabetic retinopathy.

### SUMMARY

The present invention provides a peptide comprising a glyceraldehyde-3-phosphate dehydrogenase (GAPDH) binding sequence and/or an E3 ubiquitin ligase seven in absentia homolog 1 (Siah1) binding sequence and an internalization sequence.

In a particular embodiment the peptide of the invention comprises in order from the N-terminus an internalization peptide and a GAPDH binding sequence and/or a Siah1 binding sequence.

In a particular embodiment of the invention the internalization sequence is a cationic internalization sequence, preferably a sequence comprising Seq. Id. No. 3.

In a particular embodiment the peptide of the invention comprises GAPDH binding sequence and an internalization sequence.

In a particular embodiment the peptide of the invention comprises a Siah1 binding sequence and an internalization sequence.

In a particular embodiment of the invention the GAPDH binding sequence comprises Seq. Id. No. 1.

In a particular embodiment of the invention, the Siah1 binding sequence comprises Seq. Id. No. 2.

In a particular embodiment of the invention, the N-terminus of the peptide is acetylated.

In a particular embodiment of the invention, the C-terminus of the peptide is amidated.

The invention also relates to peptides as described above for use as therapeutically active substance, in particular for the use in the treatment of diabetic retinopathy.

The invention also relates to pharmaceutical compositions comprising peptides as described above and a therapeutically inert carrier.

The invention provides a vector comprising a nucleic acid sequence encoding peptides as described above.

The invention provides a host cell comprising the vector as described above.

### BRIEF DESCRIPTION OF THE FIGURES

FIG. 1A and FIG. 1B: High glucose causes an upregulation of Siah1 total protein. hRP treated with high glucose (25 mM D-glucose) for 48 hrs have increased Siah1 total protein levels when compared to cells treated with either normal glucose (5 mM) or L-glucose (25 mM) (osmotic control) (A) (FIG. 1A). Quantification of three independent experiments is demonstrated in FIG. 1B.

FIG. 2A, FIG. 2B, FIG. 2C, FIG. 2D, FIG. 2E, FIG. 2F, and FIG. 2G: High glucose leads to an increase in the association between GAPDH and Siah1. Cells were treated with normal glucose (5 mM), L-glucose (25 mM) or high glucose (25 mM) for 48 hrs. hRP treated with high glucose have higher levels of GAPDH associated with Siah1 when compared to cells treated with either normal or L-glucose (FIG. 2A). Inhibition of the GAPDH/Siah1 pathway with either 10 μM Siah1 siRNA (FIG. 2C) or 1 μM GAPDH/Siah1 blocking TAT-FLAG peptides (FIG. 2E) inhibits high glucose-induced GAPDH/Siah1 association. Quantification of three independent experiments is shown in FIG. 2B, FIG. 2D, and FIG. 2F. High glucose-induced GAPDH/Siah 1 association was also increased in hRP nuclear fractions and nuclear accumulation was blocked by treating cells with Siah1-directed siRNA (FIG. 2G).

FIG. 3A, FIG. 3B, FIG. 3C, FIG. 3D, FIG. 3E, and FIG. 3F: High glucose causes GAPDH nuclear translocation. Nuclear levels of GAPDH are significantly increased in hRP treated with high glucose (25 mM) for 48 hrs when compared to cells treated with normal (5 mM) or L-glucose (25 mM) (FIG. 3A). Treatment with Siah1 siRNA inhibits high glucose-induced GAPDH nuclear translocation (FIG. 3C). Translocation of GAPDH can also be prevented by inhibiting the GAPDH/Siah1 binding sites using TAT-FLAG peptides (FIG. 3E). Quantification of three independent experiments is shown in FIG. 3B, FIG. 3D, and FIG. 3F.

FIG. 4A, FIG. 4B, FIG. 4C, FIG. 4D, FIG. 4E and FIG. 4F: Immunocytochemical analysis of GAPDH nuclear translocation. HRP were treated with (FIG. 4A) no primary control (FIG. 4B) normal glucose (5 mM), (FIG. 4C) L-glucose (25 mM) and (FIG. 4D) high glucose (25 mM) in the presence of absence of (FIG. 4E) control peptide or (FIG. 4F) GAPDH peptide. GAPDH is shown in red, while DAPI-stained cell nuclei are shown in blue.

FIG. 5A, FIG. 5B, FIG. 5C, and FIG. 5D: Inhibition of the GAPDH/Siah1 signaling pathway blocks high glucose-induced hRP apoptosis. Cells were treated with normal glucose (5 mM), L-glucose (25 mM) or high glucose (25 mM) for 48 hrs. Caspase-3 enzymatic activity and Annexin V levels were measured as markers for apoptosis. High glucose significantly upregulated both caspase-3 enzymatic activity (FIG. 5A) and Annexin V levels (FIG. B) when compared to normal or L-glucose. High glucose-induced caspase-3 enzymatic activity induction is significantly inhibited with Siah1-directed siRNA (FIG. 5C) and GAPDH/Siah1 blocking peptides (FIG. 5D).

FIG. 6: Proposed model of the pro-apoptotic pathway GAPDH/Siah1 in high glucose-induced human retinal pericyte apoptosis. Cell stress, such as high glucose, causes an increase in nitric oxide synthesis (NOS) activity. This increase in NOS activity results in elevated cytosolic nitric oxide (NO), which causes S-nitrosylation of GAPDH. Nitrosylated GAPDH associates with Siah1, stabilizing the complex and facilitating its translocation to the nucleus. Once in the nucleus, Siah1 degrades target proteins and/or GAPDH undertakes other non-glycolytic functions resulting in cell instability and ultimately cell death.

FIG. 7: Nuclear accumulation of GAPDH in GAPDH peptide (Peptide 1) treated rmc-1 cells (24 hours). High glucose induced nuclear accumulation of GAPDH is reduced following treatment with the GAPDH peptide (Peptide 1). Trypan blue staining was done on rmc-1 cells treated with normal (5 mM) or high (25 mM) glucose and normal (5 mM) or high (25 mM) glucose with 5 μg/mL or 10 μg/mL of the GAPDH peptide for 24 hours and cells either positive or negative for nuclear accumulation of GAPDH were counted to calculate the percentage of cells positive for nuclear accumulation of GAPDH.

FIG. 8: Nuclear accumulation of GAPDH in GAPDH peptide (Peptide 1) treated rmc-1 cells (24 hours). High glucose induced nuclear accumulation of GAPDH is reduced following treatment with the Peptide 1. Immunofluorescence staining was performed on Muller cells treated with normal (5 mM) or high (25 mM) glucose and normal (5 mM) or high (25 mM) glucose with 5 μg/mL of the Peptide 1 or scrambled peptide 1 for 24 hours and immunostained with GAPDH primary antibody. Fluorescence microscopy was done and cells either positive or negative for nuclear accumulation of GAPDH were counted to calculate the percentage of cells positive for nuclear accumulation of GAPDH±SDEV. (n=3; ns=not significant).

FIG. 9: Cell death in GAPDH peptide (Peptide 1) treated rmc-1 cells (96 hours). High glucose induced cell death is reduced following treatment with the GAPDH peptide. Trypan blue staining was done on rmc-1 cells treated with normal (5 mM) or high (25 mM) glucose and normal (5 mM) or high (25 mM) glucose with 2.5 or 5 μg/mL of the GAPDH peptide (peptide 1) for 96 hours and both live and dead cells were counted to calculate the percentage of cell death.

FIG. 10: Cell death in Peptide 1 treated Muller cells (96 hours). High glucose induced cell death is reduced following treatment with the Peptide 1. Trypan blue staining was done on Muller cells treated with normal (5 mM) or high (25 mM) glucose and normal (5 mM) or high (25 mM) glucose with 5 μg/mL of the Peptide 1 or Scrambled Peptide 1 for 96 hours and both live and dead cells were counted to calculate the mean percentage of cell death±SDEV. (n=3; *=p<0.5).

FIG. 11: Siah-1 binds with the GAPDH peptide. Immunoprecipitation of FLAG sequence of GAPDH peptide (Peptide 1) was done to analyze whether GAPDH peptide (Peptide 1) is indeed binding Siah-1 in rmc-1 cells treated with normal (5 mM) or high (25 mM) glucose and normal (5 mM) or high (25 mM) glucose with 1 μg/mL of the GAPDH peptide (Peptide 1) for 24 hours. Pull Down of GAPDH Peptide (Peptide 1) and Probed against Siah-1 to Demonstrate Binding of Peptide 1 with Siah-1 in rMC following Hyperglycemia Treatment.

FIG. 12A, FIG. 12B, and FIG. 12C: TAT-FLAG peptide identification. FIG. 12A: Immunocytochemistry analysis of anti-FLAG (red) staining in human retinal pericytes (Hrp). Top left paneldemonstrates hRPs cultured in control medium with no peptide treatment. This condition serves as a measure of background FLAG fluroescence. All four panels are stained in blue with DAPI. FIG. 12B: Immunoprecipitation western blot of anti-FLAG. FLAG-BAP fusion protein is used as a positive control to confirm the functional intergrity of anti-FLAG monoclonal antibody. FIG. 12C: Cell viability assay of hRPs treated with corresponding peptide. Cells were treated with 70% methanol for 30 mins as a positive control.

FIG. 13A and FIG. 13B: Siah1 knock-down (KD) efficiency. Siah1 expression (FIG. 13A) and protein levels (FIG. 13B) are significantly reduced with 10 μM Siah1 directed siRNA oligomers. Expression levels are measured by RT-PCR and protein levels are measured by western blot analysis.

FIG. 14A and FIG. 14B: High glucose causes an increase in nitric oxide synthase (NOS) activity (FIG. 14A) and S-nitrosylation (FIG. 14B). HRPs were treated with low glucose (5 mM, 25 mM L- or D-glucose for 48 hours. FIG. 14A: NOS activity was measured using Calbiochem NOS colorimetic kit. Graph represents total nitrite (NO²⁻) and nitrate (NO³⁻) levels. FIG. 14B: Western blot analysis of S-nitrosylated proteins. Using the Pierce S-nitrosylation western blot kit, S-nitrosocysteines are selectively reduced with ascorbate for labeling with iodoTMTzero reagent. The anti-TMT antibody was used for western blot detection of the TMT-labeled proteins. Samples treated with no ascorbate serve as negative control.

FIG. 15A, FIG. 15B, FIG. 15C and FIG. 15D: GAPDH/Siah1 complex in human retinal pericytes (HRP), human retinal microvascular endothelial cell (hRMEC) and human dermal fibroblast (Hdf). Immunocytochemistry of NG2 staining (red) in hRP (top) hRMEC (middle) and hDFs (bottom). FIG. 15A: Nuclei stained with DAPI in blue. FIG. 15B: Siah1 western blot analysis. FIG. 15C: GAPDH nuclear fractions and FIG. 15D: Caspase-3 enzymatic activity activity assay of hDFs treated with high glucose for 48 hrs. High glucose (48 hrs) does not cause GAPDH nuclear translocation or cell death in hDFs or hRMECs.

### DETAILED DESCRIPTION OF EMBODIMENTS OF THE INVENTION

The term “GAPDH” is used herein to refer to native glyceraldehyde-3-phosphate dehydrogenase polypeptide from any animal, e.g. mammalian, species, including humans, and GAPDH variants. The amino acid sequence of human GAPDH polypeptide is given in Seq. Id. No 4.

The term “Siah1” is used herein to refer to native E3 ubiquitin ligase, seven in absentia homolog 1 polypeptide from any animal, e.g. mammalian, species, including humans, and Siah1 variants. The amino acid sequence of human Siah1 polypeptide is given in Seq. Id. No 5.

The term “GAPDH binding sequence” is used herein to refer to a peptide sequence binding to GAPDH polypeptide and thereby interfering and/or blocking interaction of GAPDH polypeptide with Siah1 polypeptide.

The term “Siah1 binding sequence” is used herein to refer to a peptide sequence binding to Siah1 polypeptide and thereby interfering and/or blocking interaction of Siah1 polypeptide with GAPDH polypeptide.

The term “internalization sequence” is used herein to refer to a peptide sequence leading to cellular uptake of peptides comprising such an internalization sequence.

The term “peptide sequence” as used herein refers to an amino acid sequence of up to 50 amino acids in length.

The term “amino acid” as used herein denotes an organic molecule possessing an amino moiety located at a-position to a carboxylic group. Examples of amino acids include: arginine, glycine, ornithine, lysine, histidine, glutamic acid, asparagic acid, isoleucine, leucine, alanine, phenylalanine, tyrosine, tryptophane, methionine, serine, proline. The amino acid employed is optionally in each case the L-form.

The term “vector” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The term “expression cassette” refers to a polynucleotide generated recombinantly or synthetically, including a series of specified nucleic acid elements that permit transcription of a particular nucleic acid sequence in a target cell. A recombinant expression cassette can be incorporated into a plasmid, chromosome, mitochondrial DNA, plastid DNA, virus, or nucleic acid fragment. Typically, a recombinant expression cassette portion of an expression vector includes, among other sequences, a nucleic acid sequence to be transcribed and a promoter.

As used herein, “expression” refers to the process by which a nucleic acid is transcribed into mRNA and/or to the process by which the transcribed mRNA (also referred to as a transcript) is subsequently translated into a peptide, polypeptide, or protein. The transcripts and the encoded polypeptides are individually or collectively referred to as gene products. If a nucleic acid is derived from genomic DNA, expression in a eukaryotic cell may include splicing of the corresponding mRNA.

The terms “host cell”, “host cell line”, and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “recombinant peptide” is a peptide which has been produced by a recombinantly engineered host cell. It is optionally isolated or purified.

The peptides of the invention can be produced recombinantly or synthetically by methods well known in the art.

### Pharmaceutical Compositions and Administration

Another embodiment provides pharmaceutical compositions or medicaments containing the peptides of the invention and a therapeutically inert carrier, diluent or excipient, as well as methods of using the peptides of the invention to prepare such compositions and medicaments. In one example, peptides of the invention may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a peptide of the invention is formulated in an acetate buffer, at pH 5. In another embodiment, the peptides of the invention are sterile. The inventive peptides may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the inventive peptides to be administered will be governed by such considerations, and is the minimum amount necessary to show a therapeutic effect. For example, such amount may be below the amount that is toxic to normal cells, or the mammal as a whole.

### EXAMPLES

### Example 1: High Glucose Increases Siah1 Protein Levels in Human Retinal Pericytes (hRP)

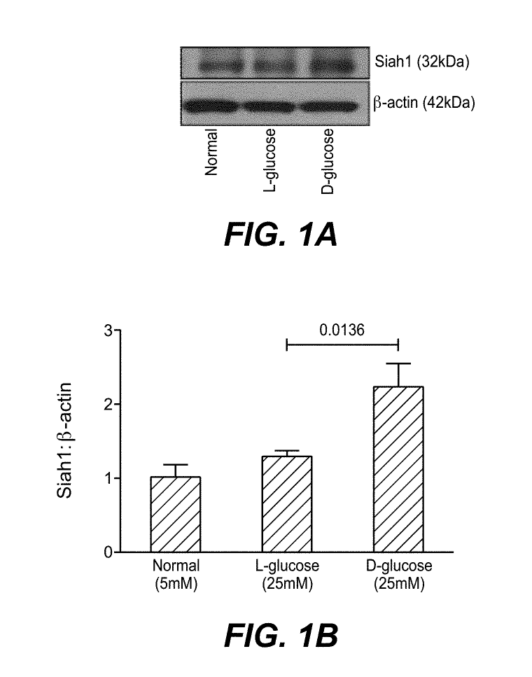

HRP were treated with normal glucose (5 mM D-glucose), osmotic control (25 mM L-glucose) or high glucose (25 mM D-glucose) for 48 hrs. Siah1 total protein increased 2-fold in cultures treated with high glucose compared to those treated with the osmotic control (p=0.0136). There was no significant difference between osmotic control and normal glucose treated cells (FIG. 1A). Quantification of three independent western blots is demonstrated in FIG. 1B.

### Example 2: High Glucose Increases the Association between GAPDH and Siah1 in hRP

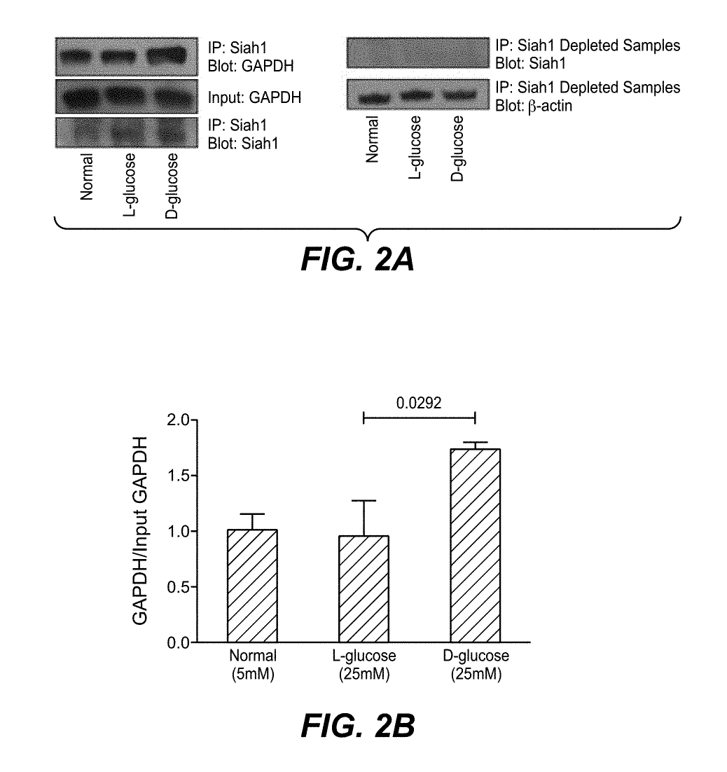

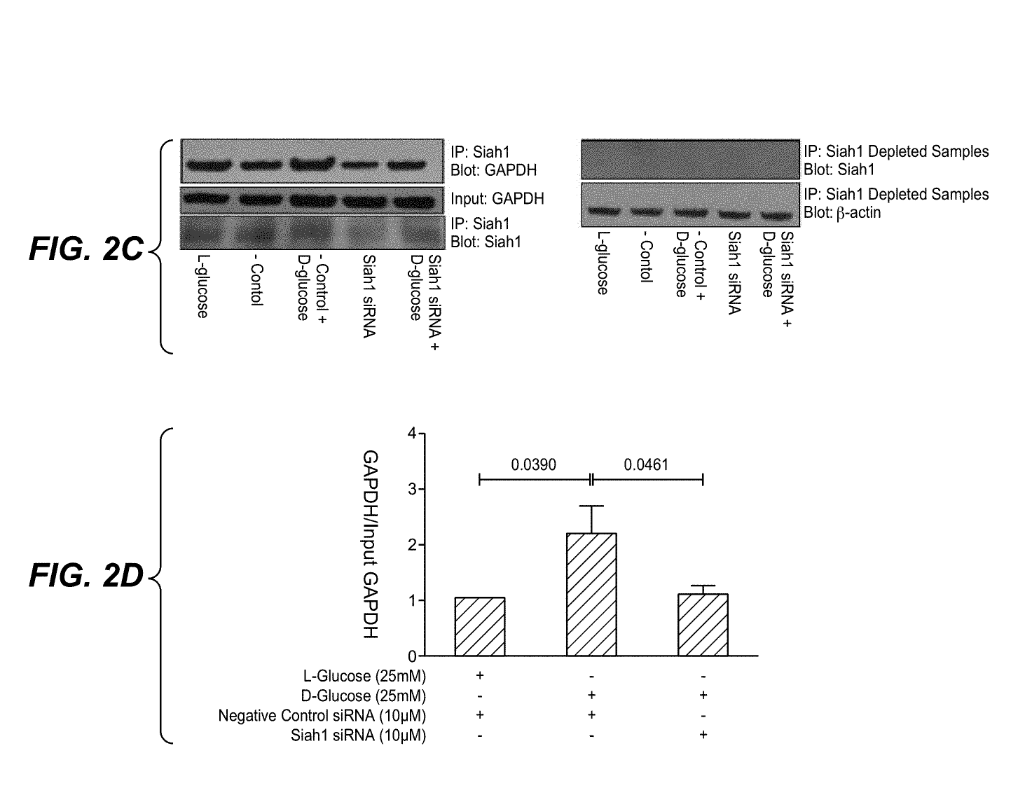

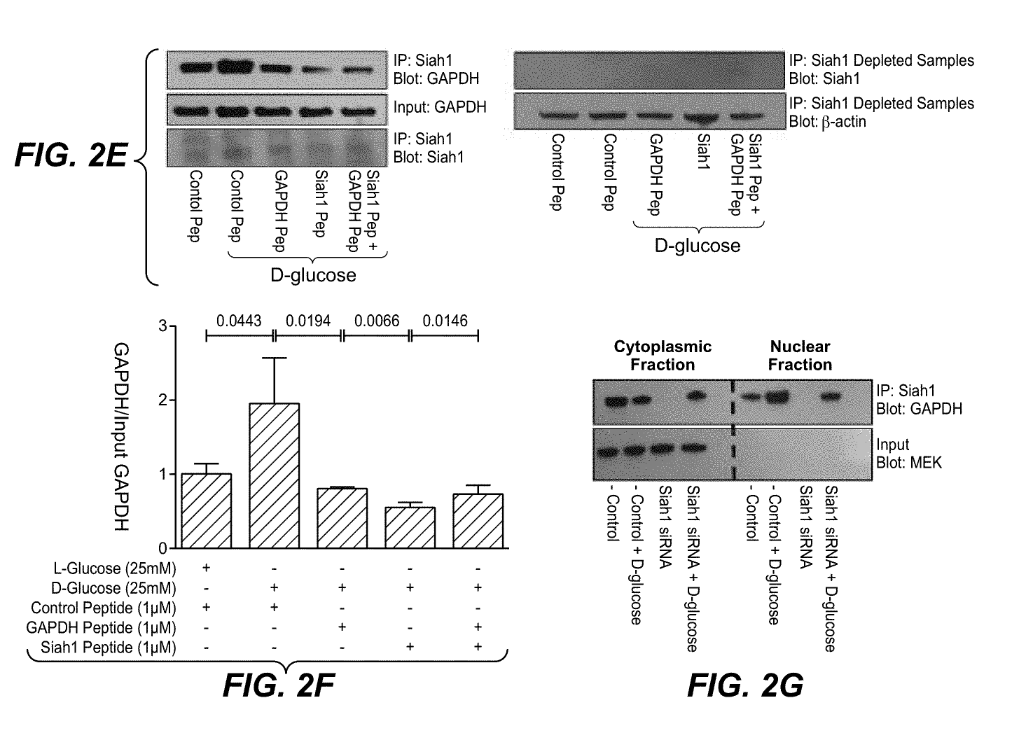

HRP were treated with normal glucose (5 mM), high glucose (25 mM) or L-glucose (25 mM) for 48 hrs. Pull down assays were performed and are described as follows: immunoprecipitation (IP) with anti-Siah1, followed by western blot (WB) analysis of the immuno-complexes with anti-GAPDH, revealed a 1.5-fold increase in GAPDH/Siah1 association in high glucose-treated cells compared to those treated with the osmotic control (p=0.0292) (FIG. 2A). Quantification of three independent western blots is demonstrated in FIG. 2B.

### Example 3: Siah1 Knockdown and Site-Specific Blocking Peptides Mitigate High Glucose-Induced GAPDH/Siah1 Association

HRP were treated with normal, osmotic control or high glucose plus 10 μM negative control siRNA, 10 μM Siah1-directed siRNA, 1 μM TAT-FLAG Control, 1 μM TAT-FLAG GAPDH peptide, 1 μM TAT-FLAG Siah1 peptide or 1 μM GAPDH+1 μM Siah1 peptides. Pull down assays were performed as described above. Our GAPDH peptide was designed to block the GAPDH binding site on Siah1 and the Siah1 peptide was designed to block the Siah1 binding site on GAPDH (Supplemental Table 1). High glucose significantly increased GAPDH/Siah1 association (p=0.0390) and this association was significantly reduced by Siah1 siRNA (p=0.0461) (FIGS. 2C, D). The GAPDH (p=0.0194) or Siah1 peptide (p=0.0066), or the combination of both (p=0.0146), significantly inhibited high glucose-induced GAPDH/Siah1 association, as well (FIGS. 2E, F). High glucose induced GAPDH/Siah1 association is also increased in hRP nuclear fractions and nuclear accumulation can be blocked by treating cells with Siah1-directed siRNA (FIG. 2G).

### Example 4: High Glucose Increases GAPDH Nuclear Translocation in hRP. Siah1 siRNA, or GAPDH/Siah1-Specific Peptides Block High Glucose-Induced GAPDH Nuclear Translocation

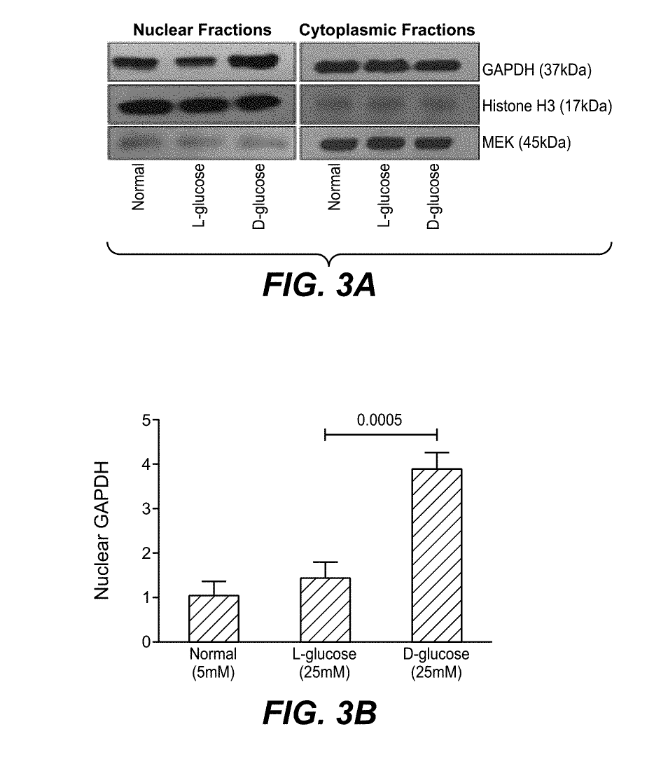

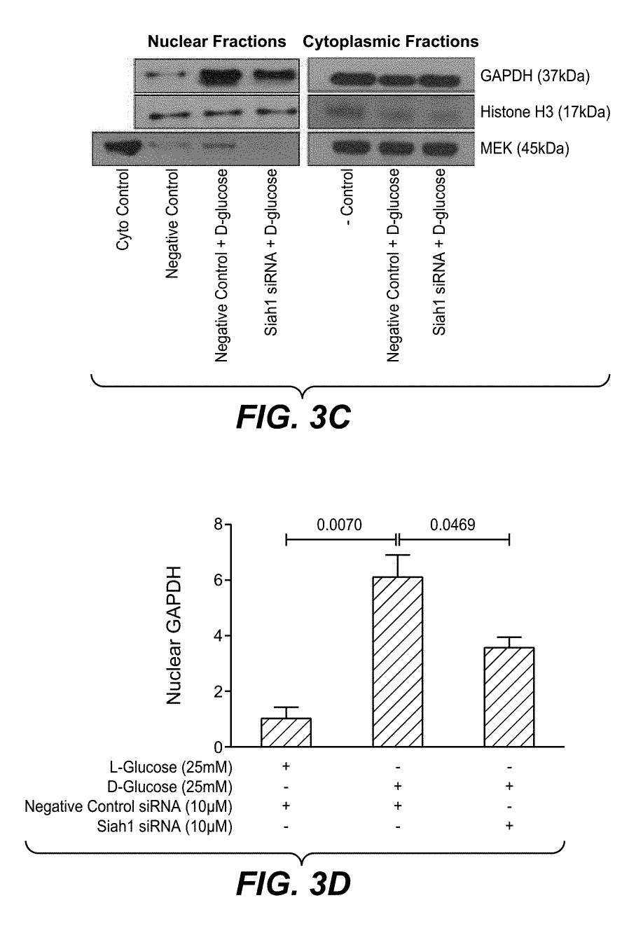

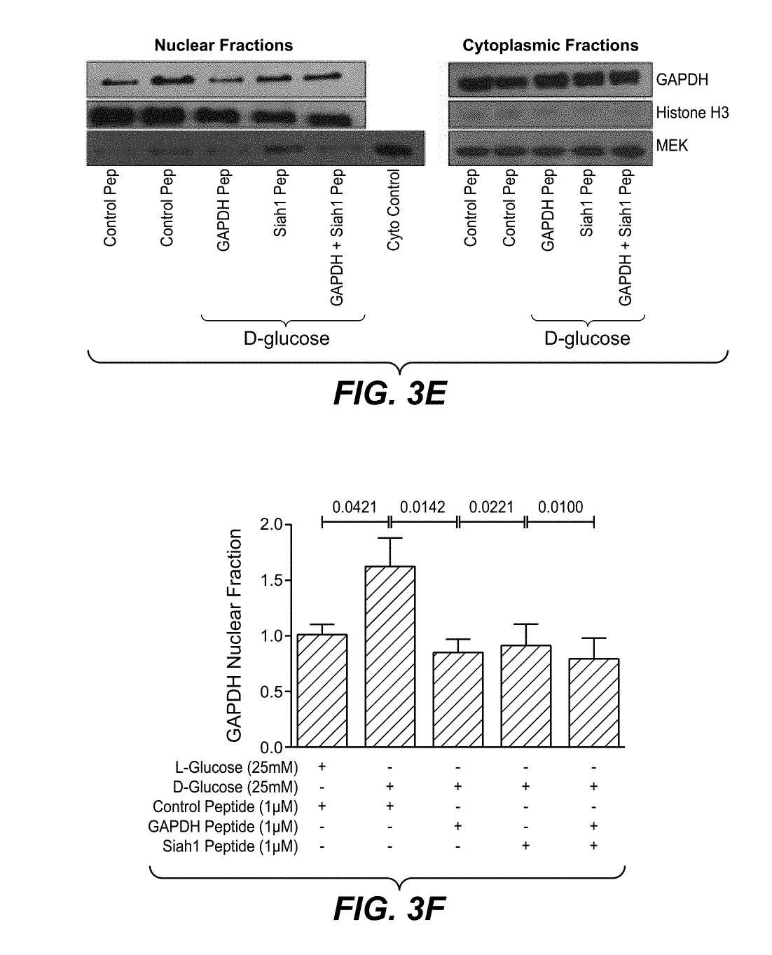

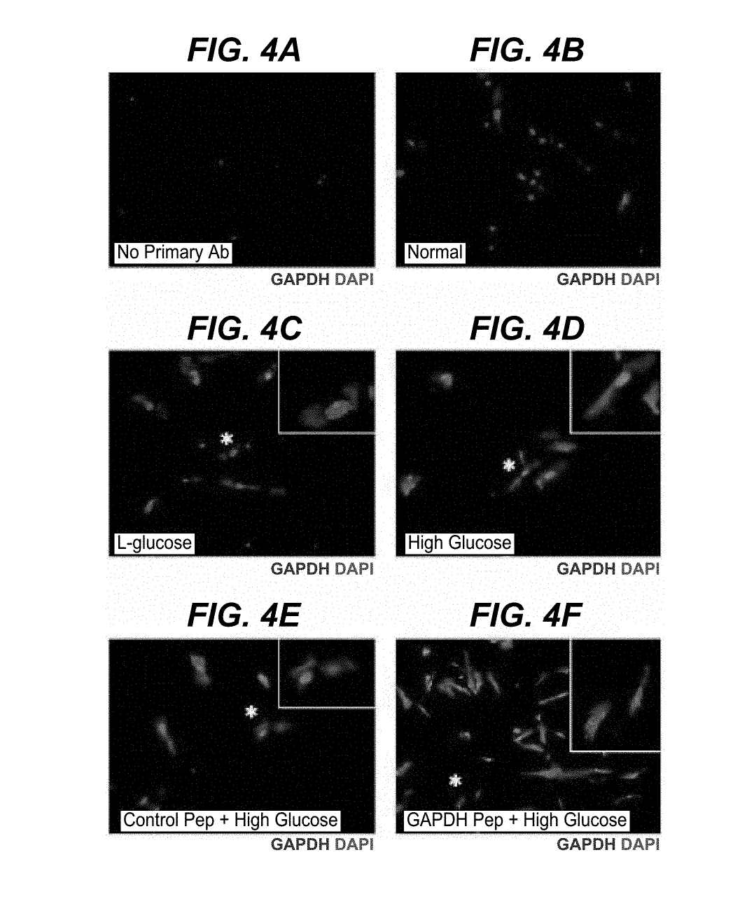

After 48 hrs of treatment with normal glucose, osmotic control or high glucose, cell lysates were prepared and separated into cytoplasmic and nuclear fractions. Each fraction was then subjected to GAPDH, MEK and Histone H3 western blot analysis. MEK and Histone H3 were used as control antigens to assess the purity of the cytoplasmic and nuclear fractions, respectively. High glucose treatment caused significant accumulation of nuclear GAPDH when compared to either normal glucose or osmotic control (p=0.0005) (FIGS. 3A, B). Siah1 siRNA (10 μM) inhibited high glucose-induced nuclear accumulation of GAPDH (p=0.0469) (FIGS. 3C, D). The GAPDH (p=0.0142) or Siah1 peptide (p=0.0221) or combination of both peptides (p=0.0100) significantly inhibited high glucose-induced GAPDH nuclear translocation (FIGS. 3E, F). GAPDH nuclear translocation was also assayed by immunocytochemical analysis, which demonstrated that translocation was induced by high glucose (FIG. 4D) and this induction was inhibited by 1 μM GAPDH peptide (FIG. 4F).

### Example 5: High Glucose Causes Human Retinal Pericyte Apoptosis by a GAPDH/Siah1-Dependent Pathway

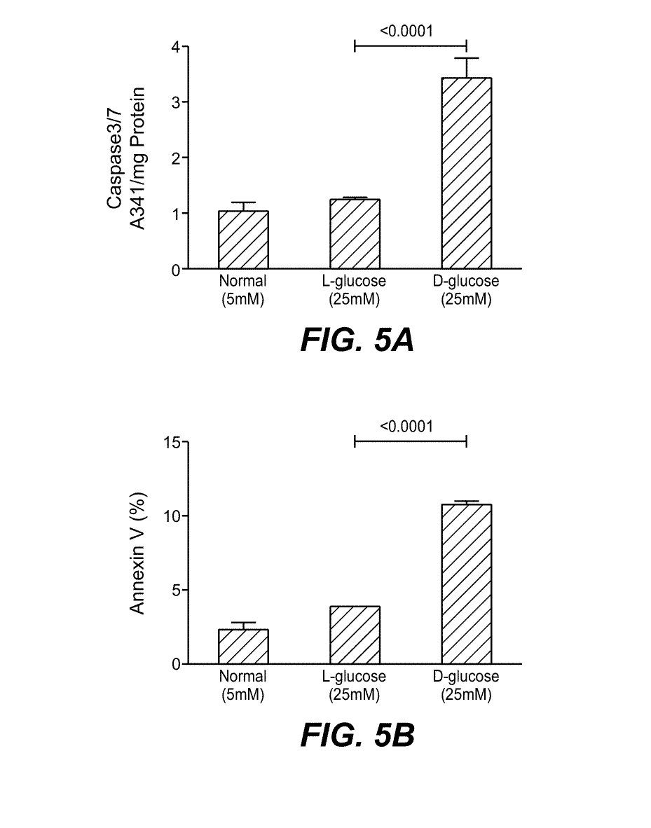

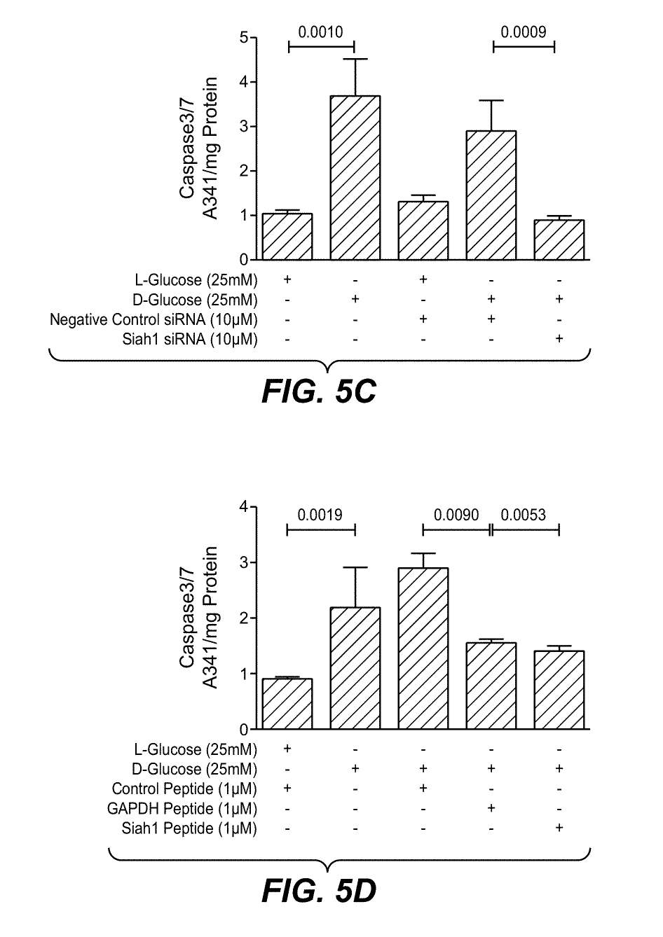

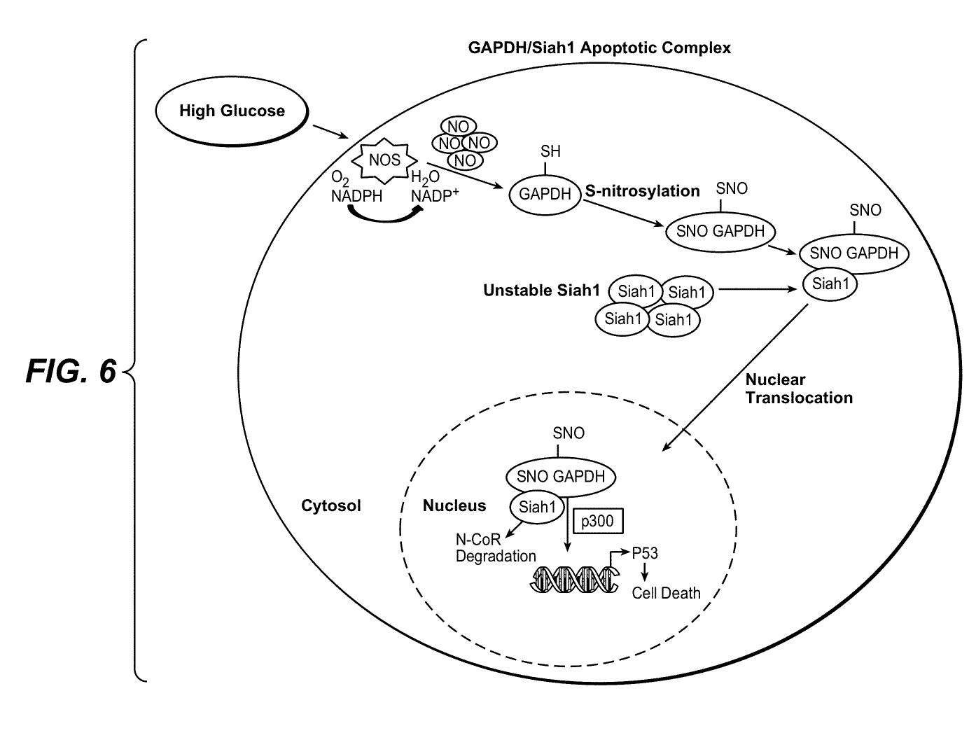

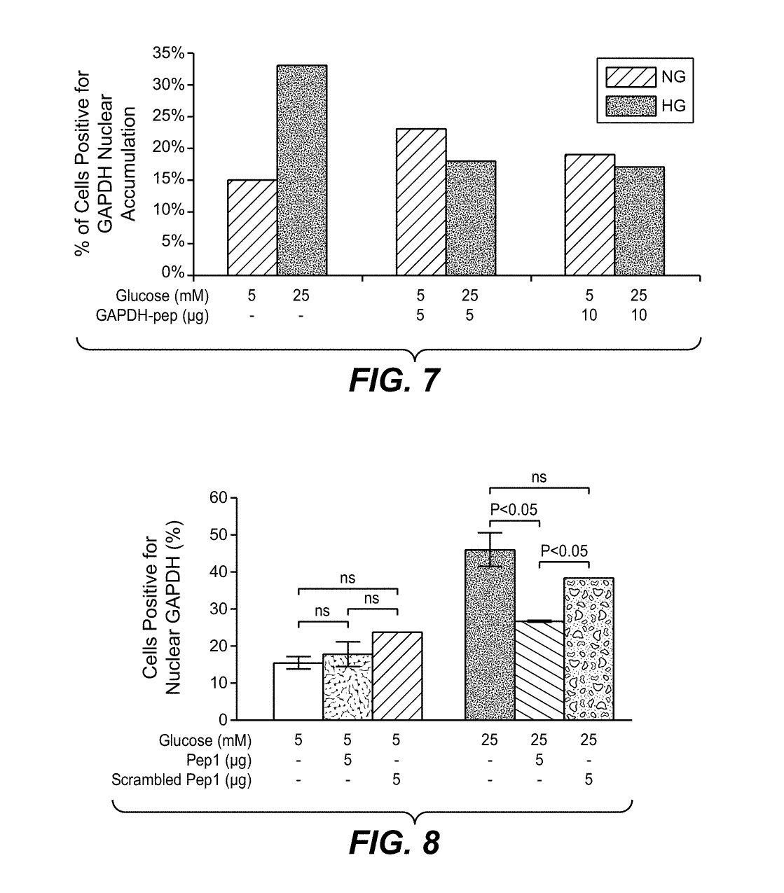

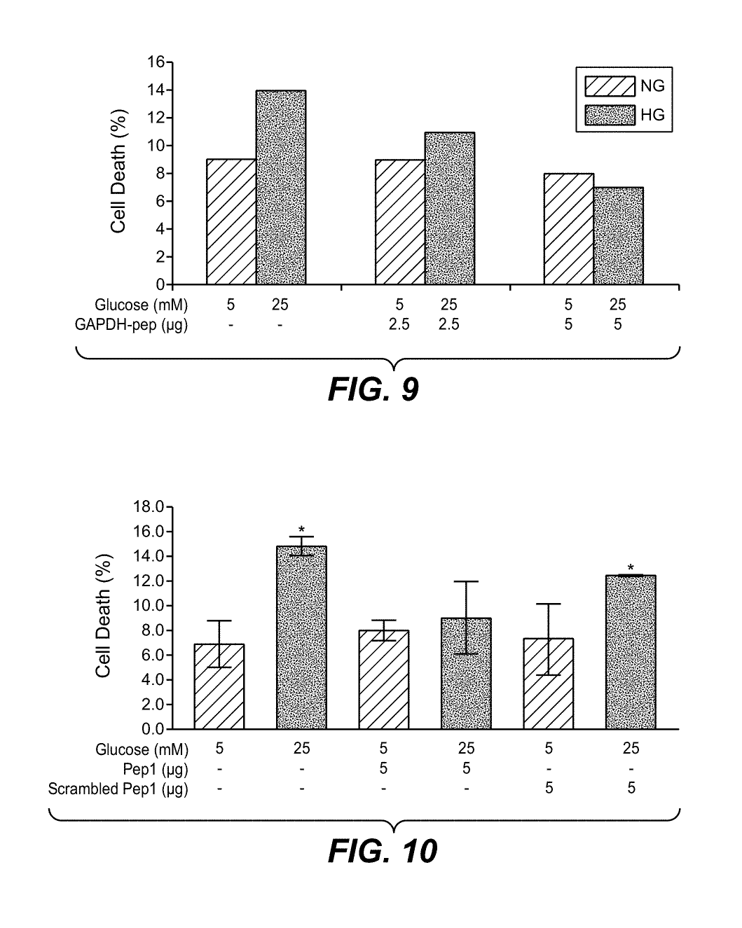

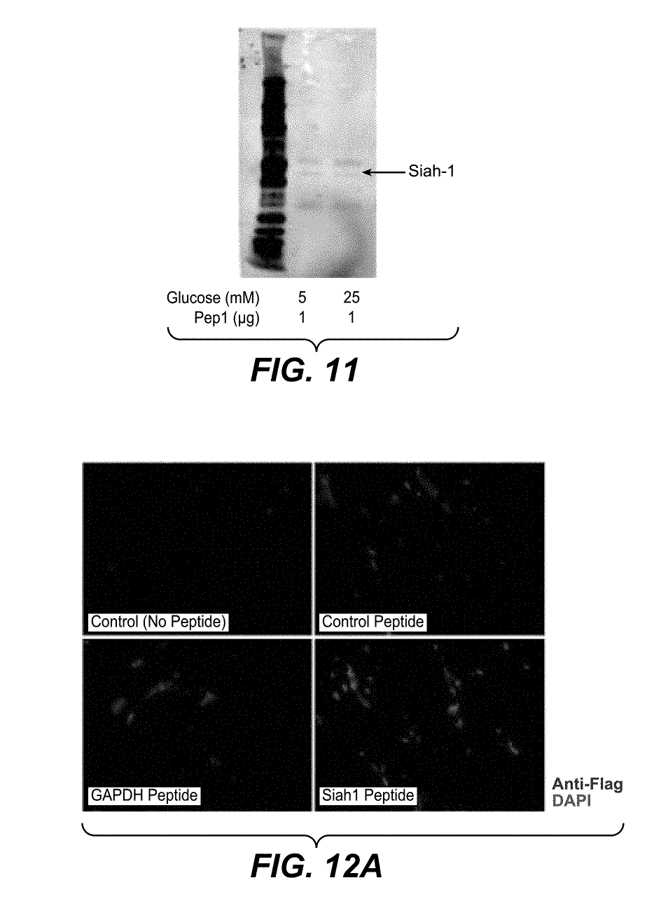

HRP were treated with normal glucose, L-glucose or high glucose for 48 hrs-72 hrs. Cell death is evident after 48 hrs of high glucose treatment and it is significantly increased after 72 hrs. Treatment with 25 mM D-glucose for 72 hrs resulted in a 3-fold increase in caspase-3-enzymatic activity, a common marker of apoptosis (p<0.0001) (FIG. 5A). High glucose exposure also caused a significant increase in Annexin V levels, another measure of apoptosis-specific cell death (p<0.0001) (FIG. 5B;). Siah1 siRNA significantly blocked this high glucose-induced apoptosis (p=0.0009) (FIG. 5C). Furthermore, GAPDH and Siah1 blocking peptides inhibited high glucose-induced hRP apoptosis (FIG. 5D Control Peptide p=0.0019, GAPDH peptide p=0.0090, Siah1 peptide p=0.0053).

A novel pro-apoptotic pathway initiated by the interaction between glyceraldehyde-3-phosphate dehydrogenase (GAPDH) and the E3 ubiquitin ligase, seven in absentia homolog 1 (Siah1), was recently identified in ocular tissues.

The inventors of the present invention examined the involvement of the GAPDH/Siah1 interaction in human retinal pericyte (hRP) apoptosis. HRP were cultured in 5 mM normal glucose, 25 mM L- or D-glucose for 48 hrs (osmotic control and high glucose treatments, respectively). Siah1 siRNA was used to downregulate Siah1 expression. TAT-FLAG GAPDH and/or Siah1 peptides were used to block GAPDH and Siah1 interaction. Co-immunoprecipitation assays were conducted to analyze the effect of high glucose on the association of GAPDH and Siah1. Apoptosis was measured by Annexin V staining and caspase-3 enzymatic activity assay. High glucose increased Siah1 total protein levels, induced the association between GAPDH and Siah1, and led to GAPDH nuclear translocation. The inventors' findings demonstrate that dissociation of the GAPDH/Siah1 pro-apoptotic complex can block high glucose-induced pericyte apoptosis, widely considered a hallmark feature of DR.

### Methods

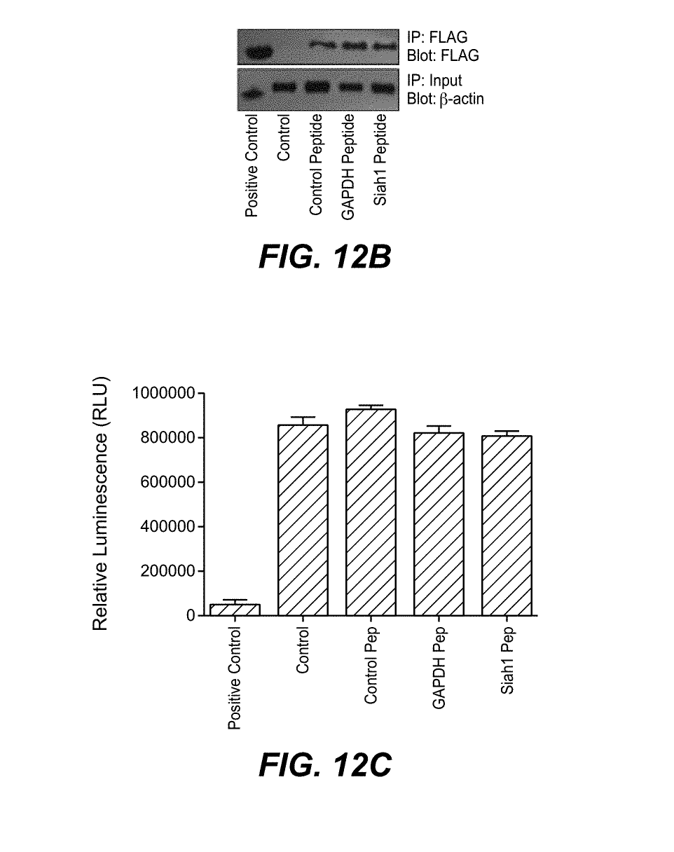

HRP Treatment: Primary cultures of human retinal pericytes (hRP) (Cell Systems; Kirkland, Wash.) were seeded into tissue culture flasks coated with attachment factor (Cell Signaling; Danvers, Mass.). HRP were grown and cultured in Dulbecco's modified Eagle's medium normal glucose (5.5 mM DMEM 1×, Life Technologies; Carlsbad, Calif.) supplemented with 10% FBS, and cell growth supplements, including antibiotics (Lonza; Basel). All cultures were incubated at 37° C., 5% CO₂ and 95% relative humidity (20.9% oxygen). Passages 5 to 7 were used for all experiments. HRP identity was confirmed by immunoreactivity of neuron glial 2 (NG2) (EMD Millipore; Temecula, Calif.). At 80% confluence hRP were treated with 10% FBS medium containing normal D-glucose (5.5 mM), high D-glucose (25 mM Sigma; St. Louis, Mo.) or L-glucose (25 mM Acros Organics; Geel, Belgium), which served as an osmotic control. For TAT-FLAG peptide treatment, 1 μM of control peptide, 1 μM GAPDH peptide and/or 1 μM Siah1 peptide was added to Hanks Balanced Salt Solution (Life Technologies; Carlsbad, Calif.). GAPDH peptide competitively blocks the GAPDH binding site on Siah1 and the Siah1 peptide competitively blocks the Siah1 peptide on GAPDH. Peptide solution was incubated at 37° C. for 30 mins before being added to each well. Cells were incubated with each peptide solution for 2 hrs before experimental treatments were added. In cases where peptides were used in combination, each original concentration was used for each peptide. The N-terminal of each TAT-peptide is acetylated and the C-terminal is amidated; these modifications ensure proper cell entry and prevent degradation once inside the cell. A FLAG tag peptide sequence enables detection and quantification of these peptides (FIG. 12).

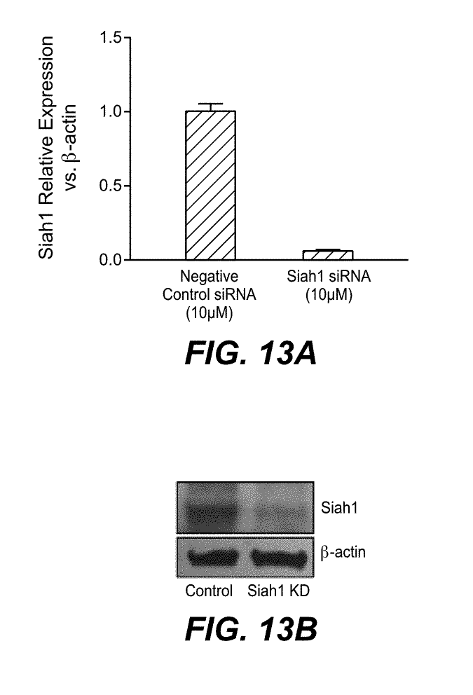

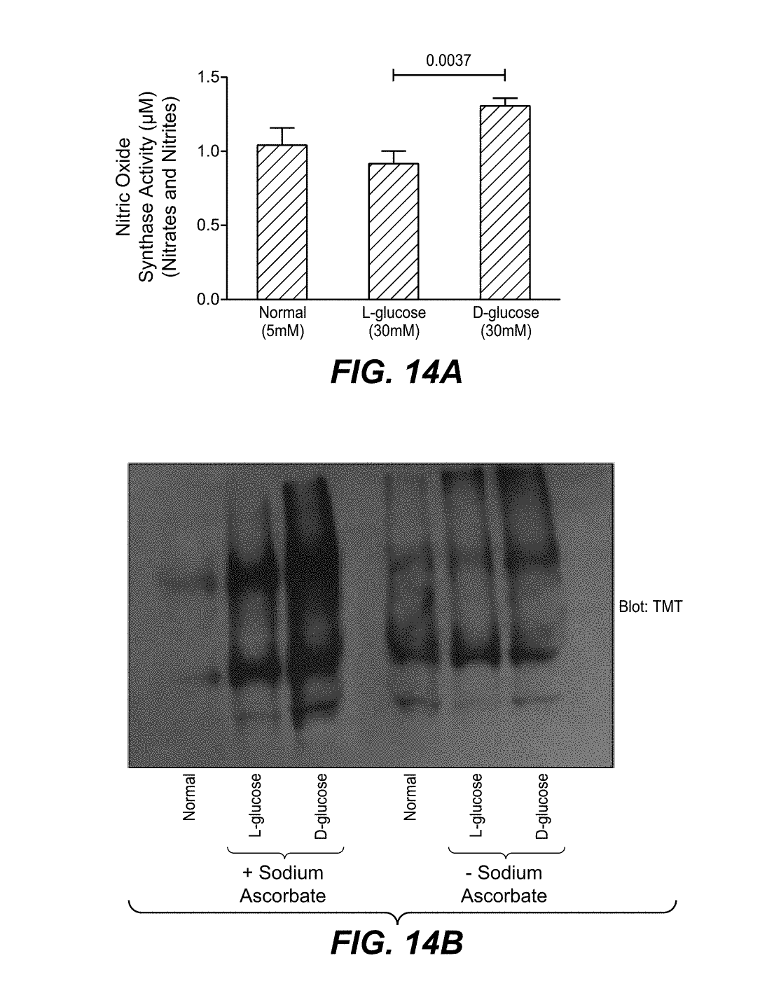

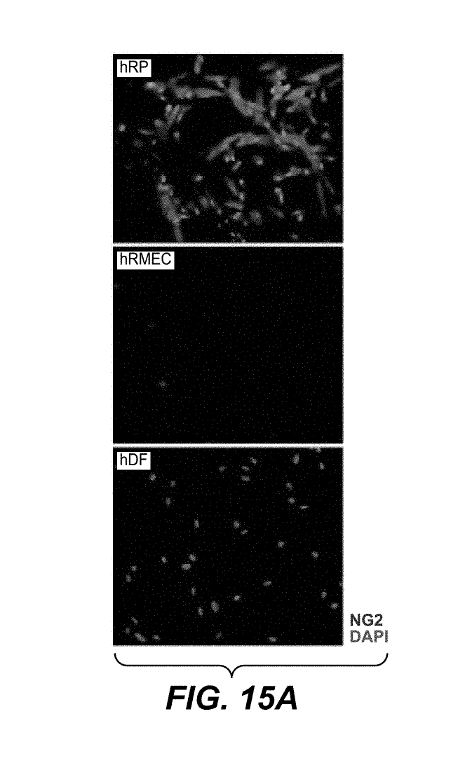

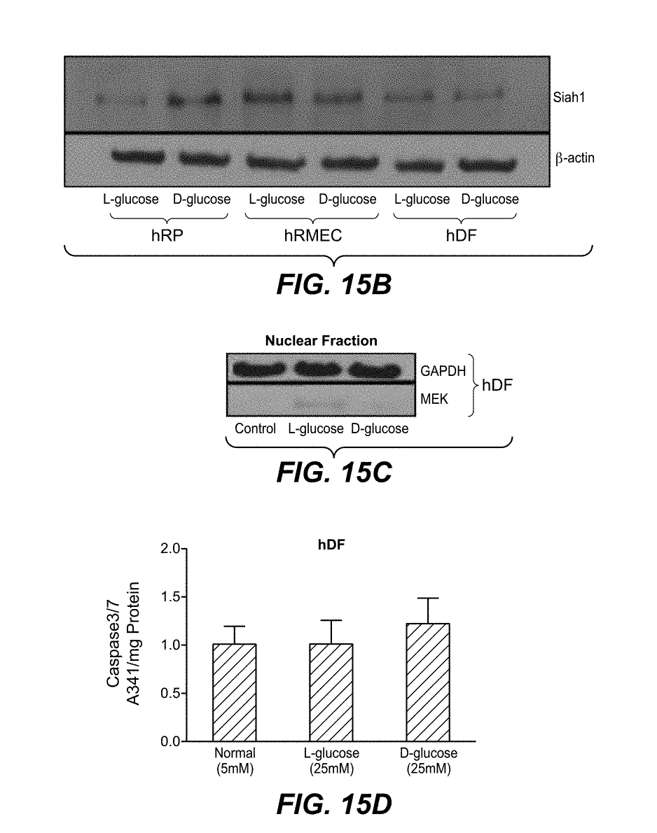

HRP Transfection: For siRNA transfection, hRP were cultured in 6-well dishes and lml of fresh media was added to each well 30 mins prior to treatment. For each well, 10 μM siRNA oligomers (negative control siRNA or Siah1-directed siRNA) (siRNA sequence identification sc-37495A, B and C, Santa Cruz; Dallas, Tex.), 9 μl Targefect Solution A (Targetingsystems; El Cajon, Calif.), and 18 μl Virofect (Targetingsystems) were added to 250 μl Optimem (Life Technologies) in a separate tube, and inverted between the addition of each reagent. Mixed reagents were incubated at 37° C. for 25 mins before being added to cultured hRP. Cells were incubated with transfection reagents for 12 hrs, before being washed and treated with fresh media. Experimental treatments began 24 hrs post-transfection. Knockdown efficiency and other quality control aspects of our siRNA experiments are shown in FIG. 13.

Nuclear Fractionation and Western Blot Analysis: HRP were treated as necessary. Cells were harvested using TrypLE Express (Life Technologies), and lysed using radioimmunoprecipitation assay (RIPA) buffer (Qiagen; Limburg, Netherlands). The NE-PER nuclear and cytoplasmic extraction reagents (Thermo Scientific; Nashville, Tenn.) were used to separate lysates into cytosolic and nuclear fractions. Samples were equilibrated for total protein concentration, subjected to 10% SDS/PAGE, and gels were transferred to nitrocellulose membranes using the iBlot system (Life Technologies). Membranes were blocked in 5% milk (for (β-actin (Thermo Scientific) and GAPDH (Abcam; Cambridge, UK) immunoblots) or 5% BSA (for Siah1 (Santa Cruz), H3 (Cell Signaling), MEK (Cell Signaling) immunoblots) probed with appropriate primary antibody (anti-(β-actin 1:3000, anti-GAPDH 1:1000, anti-Siah1 1:250, anti-Histone H3 and anti-MEK 1:750). Blots were then labeled with horseradish-peroxidase conjugated secondary antibodies diluted at 1:2000 (GAPDH, MEK and Histone H3; anti-rabbit, Siah1; anti-goat and (β-actin; anti-mouse). MEK and Histone H3 served as cytoplasmic and nuclear fractionation control. (β-actin was used to determine total protein concentration. Membranes were incubated in Pierce ECL western blotting substrate and developed using ChemiDoc MP (Bio-Rad; Hercules, Calif.). At least three independent experiments were used to generate western blot quantification graphs. Blots were quantified using the ImageJ 1.47v software.

Co-Immunoprecipitation Assays: HRP were treated as necessary and lysed using the Pierce IP Lysis Buffer. Equal amounts of protein (1000 μg) from each sample were mixed with 10 μg of anti-Siah1 antibody overnight at 4° C. Pierce Protein A/G Magnetic Beads were pre-cleared and added to the antigen sample/antibody mixture at room temperature for 1 hr. Beads were collected with a magnetic stand and eluted using 50 μl 4× SDS-PAGE reducing sample buffer at 100° C. for 10 mins. The immuno-complexes were then subjected to Western blot analysis. Siah1-depleted samples served as controls for total pull down of Siah1 from each lysate. Independent quality control experiments were performed in order to validate efficiency of the Siah1 immunoprecipitation (data not shown).

Immunocytohchemical analysis. HRP were cultured on multi-well glass slides and cells were permeabilized with 0.1% Triton-X100 in PBS for 30 mins and blocked with 1.5% BSA in PBST overnight at 4° C. Cells were incubated with anti-GAPDH primary antibody (Abcam) overnight at 4° C. After incubation with primary antibody (1:100), cells were washed and incubated with secondary antibody for 1 hr at room temperature. Cells were then washed in PBST and 40,6-diamidino-2-phenylindole (DAPI) stain was applied (Sigma). Last, cells were washed and embedded using Fluorogel with Tris buffer (Electron Microscopy Science, Hatfield, Pa., USA) and examined by fluorescence microscopy (Olympus AX70; Tokyo, Japan).

Apoptosis Measurements: All apoptosis measurements were taken after 72 hrs of appropriate treatment. Annexin V-FITC staining was one of the methods used to assay apoptosis. Briefly, cell pellets were resuspended in Annexin V binding buffer (Biolegend; San Diego, Calif.). Annexin V (Life Techonologies) and 7-AAD viability stain (Biolegend) was added to each sample for 15 mins at room temperature. Samples were quantified using flow cytometry analysis performed at Vanderbilt's Flow Cytometry Shared Resource core laboratory. Apoptosis was also assayed by measuring Caspase-3 enzymatic activity. Activity was quantified using the EnzChek Caspase-3 Assay Kit (Life Technologies). Samples were incubated with 7-amino-4-methylcoumarin-derived substrate, Z-DEVD-AMC, for 1 hr. Fluorescence emission at 440 nm was measured 2 hrs later.

Statistics: Data were analyzed with commercial software (GraphPad Prism 6; La Jolla, Calif.) using ANOVA with Fisher's LSD post hoc analysis. Values of p<0.05 were considered statistically significant.

1. A peptide comprising a glyceraldehyde-3-phosphate dehydrogenase (GAPDH) binding sequence and/or an E3 ubiquitin ligase seven in absentia homolog 1 (Siah1) binding sequence and an internalization sequence.

2. The peptide of claim 1 comprising in order from the N-terminus an internalization peptide and a GAPDH binding sequence and/or a Siah1 binding sequence.

3. The peptide of claim 1 or 2, wherein the internalization sequence is a cationic internalization sequence, preferably a sequence comprising Seq. Id. No. 3.

4. The peptide of claims 1 to 3 comprising a GAPDH binding sequence and an internalization sequence.

5. The peptide of claims 1 to 3 comprising a Siah1 binding sequence and an internalization sequence.

6. The peptide of claims 1 to 4, wherein the GAPDH binding sequence comprises Seq. Id. No. 1.

7. The peptide of claim 1 to 3 or 5, wherein the Siah1 binding sequence comprises Seq. Id. No. 2.

8. The peptide of claims 1 to 7, wherein the N-terminus of the peptide is acetylated.

9. The peptide of claims 1 to 8, wherein the C-terminus of the peptide is amidated.

10. A peptide of claims 1 to 9 for use in the treatment or prophylaxis of diabetic retinopathy.

11. Use of the peptide of claims 1 to 10 for the preparation of a medicament for the treatment or prophylaxis of diabetic retinopathy.

12. A pharmaceutical formulation comprising a peptide of claims 1 to 9.

13. A method for the treatment or prophylaxis of diabetic retinopathy comprising administering an effective amount of a peptide of claims 1-9 to a subject.

14. A vector comprising a nucleic acid sequence encoding the peptide of claims 1 to 9.

15. A host cell comprising the vector of claim 14.
